(19) United States
(12) Patent Application Publication
(10) Pub. No.: US 2020/0226752 A1
(43) Pub. Date: Jul. 16, 2020

(54) APPARATUS AND METHOD FOR PROCESSING MEDICAL IMAGE

(71) Applicant: Samsung Electronics Co., Ltd., Suwon-si (KR)

(72) Inventors: Dongjae Lee, Suwon-si (KR); Semin Kim, Suwon-si (KR); Jonghwan Min, Suwon-si (KR); Jeongyong Song, Suwon-si (KR); Jongha Lee, Suwon-si (KR)

(73) Assignee: Samsung Electronics Co., Ltd., Suwon-si (KR)

(21) Appl. No.: 16/739,885

(22) Filed: Jan. 10, 2020

(30) Foreign Application Priority Data
Jan. 16, 2019 (KR) 10-2019-0005857

(51) Int. Cl. G06T 7/00; A61B 5/00

(52) U.S. Cl. CPC G06T 7/0012; A61B 5/4842; G06T 2207/20081; G06T 2207/30061; G06T 2207/30096; G06T 2207/10072; G06T 2207/20084

(57) ABSTRACT

A medical image processing apparatus includes: a data acquisition unit configured to acquire at least one normal medical image and at least one abnormal medical image; and one or more processors configured to perform first processing for generating at least one first medical image by using a neural network and second processing for determining whether the at least one first medical image is a real image, based on the at least one abnormal medical image, wherein the first processing includes generating a virtual lesion image based on a first input and generating the at least one first medical image by synthesizing the virtual lesion image with the at least one normal medical image, and the one or more processors are further configured to train the neural network used in the first processing, based on a result of the second processing.

### CROSS-REFERENCE TO RELATED APPLICATION

This application is based on and claims priority under 35 U.S.C. § 119 to Korean Patent Application No. 10-2019-0005857, filed on Jan. 16, 2019, in the Korean Intellectual Property Office, the disclosure of which is incorporated by reference herein in its entirety.

### BACKGROUND

### 1. Field

The disclosure relates to an apparatus and method for processing a medical image, a training apparatus for training a neural network by using a medical image generated by the apparatus for processing a medical image, and a medical imaging apparatus employing the trained neural network.

### 2. Description of Related Art

A medical imaging apparatus generates a medical image by capturing an image of an object. Medical images are used for diagnostic purposes, and various research has recently been conducted into using a trained model in medical image-based diagnosis. The performance of a trained model is determined by the number and quality of training data, a learning algorithm, etc., and it is important to collect a massive amount of high quality training data in order to obtain a trained model with a level of reliability higher than a predetermined level. However, because collecting a large number of medical images is a challenging task, it is difficult to create a trained model for analyzing a medical image.

### SUMMARY

Provided are an apparatus and method for generating high quality medical images to be used as training data.

Also provided are an apparatus and method for generating various medical images corresponding to disease progression stages, which are to be used as training data.

Also provided are a training apparatus for performing training with generated training data and a medical imaging apparatus employing a model trained using the generated training data.

Additional aspects will be set forth in part in the description which follows and, in part, will be apparent from the description, or may be learned by practice of the presented embodiments of the disclosure.

According to an embodiment of the disclosure, a medical image processing apparatus includes: a data acquisition unit configured to acquire at least one normal medical image and at least one abnormal medical image; and one or more processors configured to perform first processing for generating at least one first medical image by using a neural network and second processing for determining whether the at least one first medical image is a real image based on the at least one abnormal medical image. The first processing includes generating at least one virtual lesion image based on at least one first input and generating the at least one first medical image by synthesizing the at least one virtual lesion image with the at least one normal medical image, and the one or more processors are further configured to train the neural network used in the first processing based on a result of the second processing.

The at least one first input may include a random variable input.

The at least one first input may include a lesion patch image.

The one or more processors may be further configured to train, based on the result of the second processing, a second neural network that generates the at least one first medical image by synthesizing the at least one virtual lesion image with the at least one normal medical image.

The one or more processors may be further configured to train, based on the result of the second processing, a first neural network that generates the virtual lesion image based on the at least one first input.

The at least one normal medical image and the at least one abnormal medical image may be respectively chest X-ray images.

The one or more processors may be further configured to perform the second processing by using a third neural network and train the third neural network based on the result of the second processing.

The first processing may include generating a plurality of virtual lesion images corresponding to different disease progression states based on the at least one first input and generating a plurality of first medical images corresponding to the different disease progression states by respectively synthesizing the plurality of virtual lesion images with the at least one normal medical image.

The first processing may include generating a plurality of first medical images by respectively synthesizing one of the at least one virtual lesion image with a plurality of different normal medical images.

The second processing may include determining whether the at least one first medical image is a real image based on characteristics related to lesion regions respectively in the at least one abnormal medical image and in the at least one first medical image.

The one or more processors may be further configured to select the at least one abnormal medical image to be used in the second processing, based on information about the at least one first medical image generated in the first processing.

A resolution of the at least one virtual lesion image may be lower than a resolution of the at least one abnormal medical image and a resolution of the at least one first medical image.

Each of the at least one normal medical image and the at least one abnormal medical image may be at least one of an X-ray image, a CT image, an MRI image, or an ultrasound image.

According to another embodiment of the disclosure, a training apparatus is configured to train a fourth neural network that generates an auxiliary diagnostic image showing at least one of a lesion position, a lesion type, or a probability of being a lesion by using the at least one first medical image generated by the medical image processing apparatus.

According to another embodiment of the disclosure, a medical imaging apparatus displays the auxiliary diagnostic image generated using the fourth neural network trained by the training apparatus.

According to another embodiment of the disclosure, a medical image processing method includes: acquiring at least one normal medical image and at least one abnormal medical image; performing first processing for generating at least one first medical image by using a neural network; performing second processing for determining whether the at least one first medical image is a real image based on the at least one abnormal medical image; and training the neural network used in the first processing based on a result of the second processing, wherein the performing of the first processing includes generating a virtual lesion image based on at least one first input and generating the at least one first medical image by synthesizing the virtual lesion image with the at least one normal medical image.

According to another embodiment of the disclosure, a computer program is stored on a recording medium, wherein the computer program includes at least one instruction that, when executed by a processor, performs a medical image processing method including: acquiring at least one normal medical image and at least one abnormal medical image; performing first processing for generating at least one first medical image by using a neural network; performing second processing for determining whether the at least one first medical image is a real image based on the at least one abnormal medical image; and training the neural network used in the first processing based on a result of the second processing, wherein the performing of the first processing includes generating a virtual lesion image based on at least one first input and generating the at least one first medical image by synthesizing the virtual lesion image with the at least one normal medical image.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1A is an external view and block diagram of a configuration of an X-ray apparatus according to an embodiment of the disclosure, wherein the X-ray apparatus is a fixed X-ray apparatus;

FIG. 1B is an external view and block diagram of a configuration of a mobile X-ray apparatus as an example of an X-ray apparatus;

FIG. 2 is a block diagram of a configuration of a medical image processing apparatus according to an embodiment of the disclosure;

FIG. 3 illustrates operations of a processor and a neural network, according to an embodiment of the disclosure;

FIG. 4 is a diagram for explaining a procedure for performing processing for generating a first medical image, according to an embodiment of the disclosure;

FIG. 5 is a flowchart of a medical image processing method according to an embodiment of the disclosure;

FIG. 6 illustrates structures of a processor and a neural network, according to an embodiment of the disclosure;

FIG. 7 illustrates structures of a processor and a neural network, according to an embodiment of the disclosure;

FIG. 8 illustrates a form of a first input according to an embodiment of the disclosure;

FIG. 9 illustrates a process of generating a first medical image, according to an embodiment of the disclosure;

FIG. 10 illustrates a training apparatus and an auxiliary diagnostic device, according to an embodiment of the disclosure;

FIG. 11 is a block diagram of a configuration of a medical imaging apparatus according to an embodiment of the disclosure; and

FIG. 12 is a block diagram of a configuration of a medical imaging apparatus according to an embodiment of the disclosure.

### DETAILED DESCRIPTION

The principle of the disclosure is explained and embodiments of the disclosure are disclosed so that the scope of the disclosure is clarified and one of ordinary skill in the art to which the disclosure pertains may implement the disclosure. The embodiments of the disclosure may have various forms.

Throughout the specification, like reference numerals or characters refer to like elements. In the present specification, all elements of embodiments of the disclosure are not explained, but general matters in the technical field of the disclosure or redundant matters between embodiments of the disclosure will not be described. Terms ‘module’ or ‘unit’ used herein may be implemented using at least one or a combination from among software, hardware, or firmware, and, according to embodiments of the disclosure, a plurality of ‘module’ or ‘unit’ may be implemented using a single element, or a single ‘module’ or ‘unit’ may be implemented using a plurality of units or elements. The operational principle of the disclosure and embodiments of the disclosure will now be described more fully with reference to the accompanying drawings.

In the present specification, an image may include a medical image obtained by a medical imaging apparatus, such as a computed tomography (CT) apparatus, a magnetic resonance imaging (MRI) apparatus, an ultrasound imaging apparatus, or an X-ray apparatus.

Throughout the specification, the term ‘object’ is a thing to be imaged, and may include a human, an animal, or a part of a human or animal. For example, the object may include a part of a body (i.e., an organ), a phantom, or the like.

Throughout the disclosure, the expression “at least one of a, b or c” indicates only a, only b, only c, both a and b, both a and c, both b and c, all of a, b, and c, or variations thereof.

Embodiments of the disclosure may be applied to a CT image, an MR image, an ultrasound image, or an X-ray image. Although the disclosure is mainly described with respect to an example in which embodiments of the disclosure are applied to an X-ray image, it will be readily understood that the scope of the disclosure as defined by the appended claims is not limited to an embodiment of the disclosure in which an X-ray image is used but covers embodiments of the disclosure in which medical images of other modalities are used.

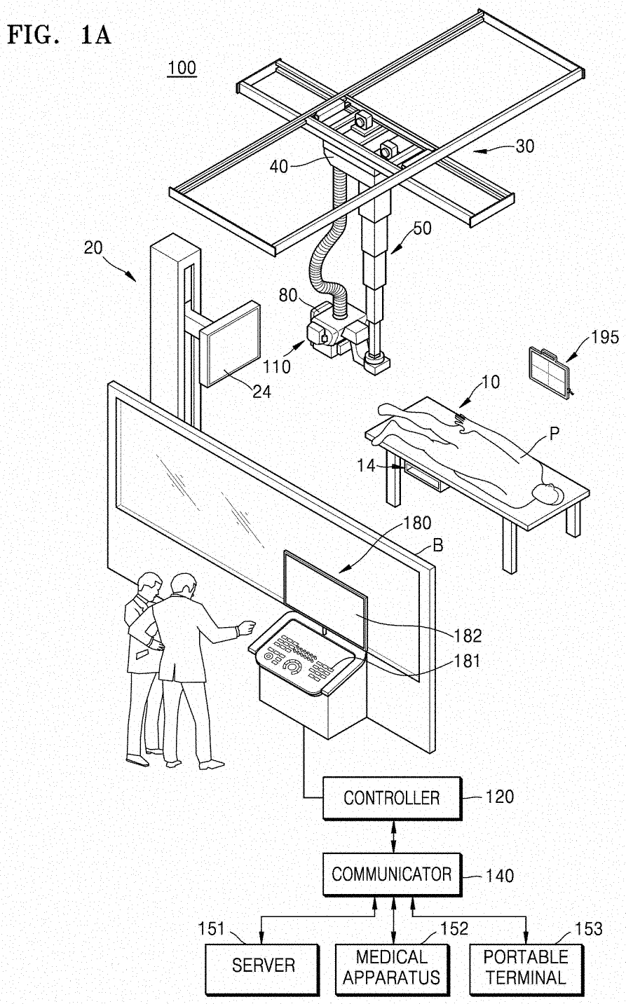

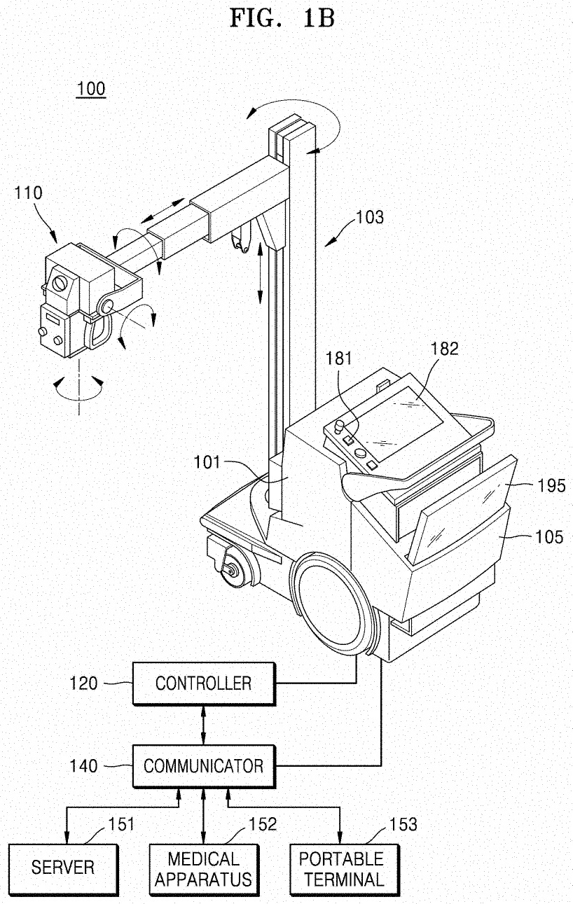

FIG. 1A is an external view and block diagram of a configuration of an X-ray apparatus 100 according to an embodiment of the disclosure. In FIG. 1A, it is assumed that the X-ray apparatus 100 is a fixed X-ray apparatus.

Referring to FIG. 1A, the X-ray apparatus 100 includes an X-ray radiation device 110 for generating and emitting X-rays, an X-ray detector 195 for detecting X-rays that are emitted by the X-ray radiation device 110 and transmitted through an object P, and a workstation 180 for receiving a command from a user and providing information to the user. The X-ray apparatus 100 may further include a controller 120 for controlling the X-ray apparatus 100 according to the received command, and a communicator 140 for communicating with an external device.

All or some components of the controller 120 and the communicator 140 may be included in the workstation 180 or be separate from the workstation 180.

The X-ray radiation device 110 may include an X-ray source for generating X-rays and a collimator for adjusting a region irradiated with the X-rays generated by the X-ray source.

A guide rail 30 may be provided on a ceiling of an examination room in which the X-ray apparatus 100 is located, and the X-ray radiation device 110 may be coupled to a moving carriage 40 that is movable along the guide rail 30 such that the X-ray radiation device 110 may be moved to a position corresponding to the object P. The moving carriage 40 and the X-ray radiation device 110 may be connected to each other via a foldable post frame 50 such that a height of the X-ray radiation device 110 may be adjusted.

The workstation 180 may include an input device 181 for receiving a user command and a display 182 for displaying information.

The input device 181 may receive commands for controlling imaging protocols, imaging conditions, imaging timing, and locations of the X-ray radiation device 110. The input device 181 may include a keyboard, a mouse, a touch screen, a microphone, a voice recognizer, etc.

The display 182 may display a screen for guiding a user's input, an X-ray image, a screen for displaying a state of the X-ray apparatus 100, and the like.

The controller 120 may control imaging conditions and imaging timing of the X-ray radiation device 110 according to a command input by the user and may generate a medical image based on image data received from an X-ray detector 195. Furthermore, the controller 120 may control a position or orientation of the X-ray radiation device 110 or mounting units 14 and 24, each having the X-ray detector 195 mounted therein, according to imaging protocols and a position of the object P.

The controller 120 may include a memory configured to store programs for performing the operations of the X-ray apparatus 100 and a processor or a microprocessor configured to execute the stored programs. The controller 120 may include a single processor or a plurality of processors or microprocessors. When the controller 120 includes the plurality of processors, the plurality of processors may be integrated onto a single chip or be physically separated from one another.

The X-ray apparatus 100 may be connected to external devices such as an external server 151, a medical apparatus 152, and/or a portable terminal 153 (e.g., a smart phone, a tablet PC, or a wearable device) in order to transmit or receive data via the communicator 140.

The communicator 140 may include at least one component that enables communication with an external device. For example, the communicator 140 may include at least one of a local area communication module, a wired communication module, or a wireless communication module.

The communicator 140 may receive a control signal from an external device and transmit the received control signal to the controller 120 so that the controller 120 may control the X-ray apparatus 100 according to the received control signal.

In addition, by transmitting a control signal to an external device via the communicator 140, the controller 120 may control the external device according to the control signal. For example, the external device may process data of the external device according to the control signal received from the controller 120 via the communicator 140

The communicator 140 may further include an internal communication module that enables communications between components of the X-ray apparatus 100. A program for controlling the X-ray apparatus 100 may be installed on the external device and may include instructions for performing some or all of the operations of the controller 120.

The program may be preinstalled on the portable terminal 153, or a user of the portable terminal 153 may download the program from a server providing an application for installation. The server that provides applications may include a recording medium where the program is stored.

Furthermore, the X-ray detector 195 may be implemented as a fixed X-ray detector that is fixedly mounted to a stand 20 or a table 10 or as a portable X-ray detector that may be detachably mounted in the mounting unit 14 or 24 or can be used at arbitrary positions. The portable X-ray detector may be implemented as a wired or wireless detector according to a data transmission technique and a power supply method.

The X-ray detector 195 may or may not be a component of the X-ray apparatus 100. The X-ray detector 195 is not a component of the X-ray apparatus 100, the X-ray detector 195 may be registered by the user with the X-ray apparatus 100. Furthermore, in both cases, the X-ray detector 195 may be connected to the controller 120 via the communicator 140 to receive a control signal from or transmit image data to the controller 120.

A sub-user interface 80 that provides information to a user and receives a command from the user may be provided on one side of the X-ray radiation device 110. The sub-user interface 80 may also perform some or all of the functions performed by the input device 181 and the display 182 of the workstation 180.

When all or some components of the controller 120 and the communicator 140 are separate from the workstation 180, they may be included in the sub-user interface 80 provided on the X-ray radiation device 110.

Although FIG. 1A shows a fixed X-ray apparatus connected to the ceiling of the examination room, examples of the X-ray apparatus 100 may include a C-arm type X-ray apparatus, a mobile X-ray apparatus, and other X-ray apparatuses having various structures that will be apparent to those of ordinary skill in the art.

FIG. 1B is an external view and block diagram of a configuration of a mobile X-ray apparatus as an example of an X-ray apparatus 100.

The same reference numerals as those in FIG. 1A denote elements performing the same functions, and thus descriptions with respect to the reference numerals in FIG. 1A will not be repeated below.

An X-ray apparatus may be implemented not only as the ceiling type as described above, but also as a mobile type. When the X-ray apparatus 100 is implemented as a mobile X-ray apparatus, a main body 101 to which an X-ray radiation device 110 is connected is freely movable, and an arm 103 connecting the X-ray radiation device 110 to the main body 101 may also be rotated and be moved linearly. Thus, the X-ray radiation device 110 may freely move in a three-dimensional (3D) space.

The main body 101 may include a holder 105 for accommodating an X-ray detector 195. Furthermore, a charging terminal capable of charging the X-ray detector 195 is provided in the holder 105 such that the X-ray detector 195 may be kept in the holder 105 while being charged.

An input device 181, a display 182, the controller 120, and a communicator 140 may be mounted on the main body 101. Image data acquired by the X-ray detector 195 may be transmitted to the main body 101 and undergo image processing before being displayed on the display 182 or being transmitted to an external device through the communicator 140.

Furthermore, the controller 120 and the communicator 140 may be provided separately from the main body 101, and only some of the components of the controller 120 and the communicator 140 may be provided in the main body 101.

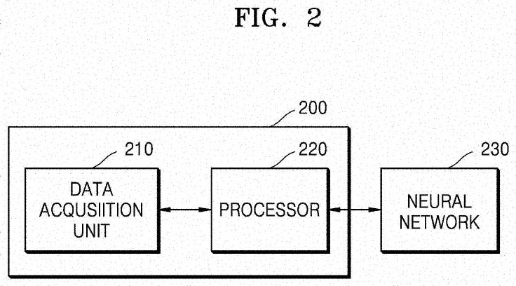

FIG. 2 is a block diagram of a configuration of a medical image processing apparatus 200 according to an embodiment of the disclosure.

According to an embodiment of the disclosure, the medical image processing apparatus 200 includes a data acquisition unit 210 and a processor 220. The processor 220 generates a first medical image by using a neural network 230. The neural network 230 may be included in the medical image processing apparatus 200 or may be provided in an external device.

The data acquisition unit 210 acquires at least one normal medical image and at least one abnormal medical image.

A normal medical image is a medical image acquired by capturing an image of a patient in which a disease or lesion is not detected. An abnormal medical image is a medical image acquired by capturing an image of a patient with a disease or lesion. In this case, the normal and abnormal medical images are real medical images obtained by actually capturing images of patients. A medical image may be determined as a normal or abnormal medical image based on diagnosis by medical personnel, a medical diagnostic imaging apparatus, etc. Information about whether a medical image is normal or abnormal one may be written to metadata related to the medical image.

The normal and abnormal medical images may each include metadata related to a patient or disease. For example, the metadata may include additional information such as an imaging protocol, a patient's age, gender, race, body weight, height, biometric information, disease information, disease history, family medical history, diagnostic information, etc.

The normal and abnormal medical images may be captured medical images of corresponding regions or organs. For example, the normal and abnormal medical images may correspond to chest images, abdominal images, or bone images. Furthermore, the normal and abnormal medical images may be medical images captured in a predefined direction. For example, the normal and abnormal medical images may be captured in a predefined direction such as front, side, or rear.

According to an embodiment of the disclosure, the normal and abnormal medical images may each have predefined characteristics. For example, predefined characteristics of a medical image may include its size and resolution, alignment of an object therein, etc.

According to an embodiment of the disclosure, the data acquisition unit 210 may correspond to a storage medium. For example, the data acquisition unit 210 may be implemented as a memory, a non-volatile data storage medium for storing data, or the like. The data acquisition unit 210 may correspond to a database for storing a medical image.

According to another embodiment of the disclosure, the data acquisition unit 210 may correspond to an input/output (I/O) device or a communicator used to acquire a medical image from an external device. Examples of an external device may include an X-ray imaging system, a computed tomography (CT) system, a magnetic resonance imaging (MRI) system, a medical data server, another user's terminal, etc. According to an embodiment of the disclosure, the data acquisition unit 210 may be connected to an external device via various wired/wireless networks such as a wired cable, a local area network (LAN), a mobile communication network, the Internet, etc. The data acquisition unit 210 may correspond to the communicator 140 described with reference to FIG. 1A or 1B.

The processor 220 may control all operations of the medical image processing apparatus 200 and process data. The processor 220 may include one or more processors. The processor 220 may correspond to the controller 120 described with reference to FIG. 1A or 1B.

The processor 220 generates a first medical image based on a first input and a normal medical image by using the neural network 230.

According to an embodiment of the disclosure, the processor 220 generates a first medical image by using the neural network 230 provided externally. To achieve this, the processor 220 may transmit the first input and the normal medical image to the neural network 230 externally provided, control an operation of the neural network 230, and receive the first medical image output from the neural network 230 For example, the neural network 230 may be located in an external server or external system connected to the medical image processing apparatus 200.

According to another embodiment of the disclosure, the neural network 230 may be provided in the medical image processing apparatus 200. According to an embodiment of the disclosure, the neural network 230 is formed as a block that is separate from the processor 220, and the processor 220 and the neural network 230 may be formed as separate integrated circuit (IC) chips. According to another embodiment of the disclosure, the neural network 230 and the processor 220 may be formed as a single block within a single IC chip.

The neural network 230 may be formed as a combination of at least one processor and at least one memory. The neural network 230 may include one or a plurality of neural networks. The neural network 230 may receive a first input and a normal medical image to output a first medical image. The processor 220 may transmit input data to the neural network 230 and acquire data output from the neural network 230. The neural network 230 may include at least one layer, at least one node, and a weight between the at least one node. The neural network 230 may correspond to a deep neural network composed of a plurality of layers.

The processor 220 performs first processing for generating at least one first medical image by using the neural network 230 and second processing for determining whether the at least one first medical image is a real image based on at least one abnormal medical image.

It is necessary to train a neural network with a large number of training data to implement a medical artificial intelligence (AI) system for generating diagnostic information related to a medical image by using the neural network. A huge number of high quality training data are required to build a medical AI system with high reliability. However, because collecting and managing the huge number of high quality training data requires large amounts of cost and efforts, this is a technical challenge for building a medical AI system. Furthermore, to train a medical AI system for acquiring information about a disease or lesion, a medical image of a patient with the disease or lesion need to be acquired as training data. However, because the number of medical images of a patient with a disease or lesion is less than the number of medical images of a normal patient, it is more difficult to acquire abnormal medical images than normal medical images. According to embodiments of the disclosure, various first medical images may be generated using a first input and a normal medical image, thereby allowing acquisition of a large number of training data.

A generative adversarial network (GAN) technique is one of the algorithms for generating a virtual image. The GAN technique may be used to generate a virtual image. However, a medical image is different from other common images in terms of its high resolution, distribution and range of gray levels, and image characteristics. Due to these differences, when virtual medical images are generated by applying a GAN technique to a medical image, the virtual medical images are of poor quality. Furthermore, a virtual medical image generated using a GAN technique differs from a real medical image in terms of its quality level.

According to embodiments of the disclosure, a virtual lesion image having a size smaller than that of a first medical image that is a final output image may be initially generated, and the first medical image of high quality may then be obtained by synthesizing the virtual lesion image with a normal medical image. Furthermore, according to embodiments of the disclosure, it is determined, based on an actually captured abnormal medical image, whether a first medical image is a real image, and the first medical image of a high quality may be obtained by training, based on a determination result, a neural network used for generating the first medical image and a neural network used for determining whether the first medical image is a real one.

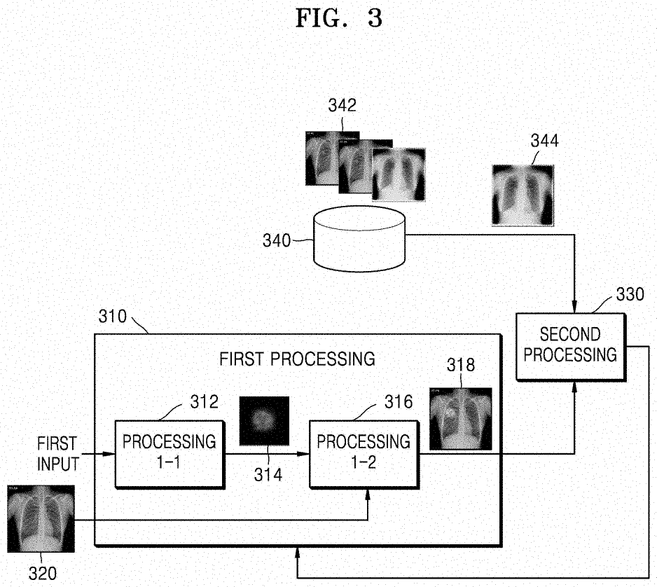

FIG. 3 illustrates operations of the processor 220 and the neural network 230 according to an embodiment of the disclosure.

When the processor 220 is configured to include the neural network 230, the processor 220 performs first processing 310 and second processing 330 shown in FIG. 3. Otherwise, when the neural network 230 is located outside the processor 220, the neural network 230 performs some operations of the first processing 310 and the second processing 330, and the processor 220 may transmit input data to the neural network 230, acquire data output from the neural network 230, and process or transmit the acquired data. Furthermore, the processor 220 may perform an operation of training the neural network 230 based on a result of the second processing 330.

Blocks of the first processing 310, processing 1-1 312, processing 1-2 316, and the second processing 330 may respectively correspond to blocks of software processing performed by executing at least one instruction. Thus, each processing block is used to represent a flow of processing and does not limit a hardware configuration. Furthermore, the blocks of the first processing 310, processing 1-1 312, processing 1-2 316, and second processing 330 may be implemented by a combination of various processors, a graphic processing unit (GPU), a dedicated processor, a dedicated IC chip, a memory, a buffer, a register, etc.

In the first processing 310, a first medical image 318 is generated based on a first input and a normal medical image 320. The first processing 310 may include the processing 1-1 312 for generating a virtual lesion image 314 and processing 1-2 316 for generating the first medical image 318 by synthesizing the virtual lesion image 314 with the normal medical image 320.

During the processing 1-1 312, the virtual lesion image 314 is generated based on the first input. The first input may define an initial value, a parameter value, etc. used to generate the virtual lesion image 314. According to an embodiment of the disclosure, the first input may be a random variable. According to another embodiment of the disclosure, the first input may be a lesion patch image. In the processing 1-1 312, the virtual lesion image 314 is generated by determining, based on the first input, a shape and a size of the virtual lesion image 314, a type of lesion and pixel values in the virtual lesion image 314, etc. According to an embodiment of the disclosure, the processing 1-1 312 may be performed to generate the virtual lesion image 314 based on the first input by using a predefined function. According to another embodiment of the disclosure, the processing 1-1 312 may be performed to generate the virtual lesion image 314 from the first input by using a first neural network. The first neural network may be implemented as a deep neural network in which a plurality of nodes and weights between the plurality of nodes are defined. The first neural network may be trained using a predetermined learning algorithm, based on a resulting value of the second processing 330

According to an embodiment of the disclosure, the first input may correspond to a random variable. In the processing 1-1 312, the virtual lesion image 314 is generated using a random variable as an initial value or parameter value. The random variable may correspond to a single value or a set of a plurality of values. One or a plurality of values contained in a random variable may be generated using a predetermined random variable generation algorithm. The number of digits and a range of values in the random variable, an interval between the values, the number of values, etc. may be predefined.

According to another embodiment of the disclosure, the first input may correspond to a lesion patch image. In the processing 1-1 312, the virtual lesion image 314 may be generated using a lesion patch image as an initial value or parameter value. A lesion patch image used as the first input may define an initial value such as a shape and type of a lesion, a pixel value distribution in the lesion, etc. The lesion patch image may correspond to a combination of types and shapes of a plurality of lesions and pixel value distributions in the lesions. The lesion patch image may be an image acquired based on a real medical image, an image acquired by deforming the real medical image, or an image generated using an algorithm for generating a lesion image. According to an embodiment of the disclosure, the virtual lesion image 314 generated by performing the processing 1-1 312 may be used again as the first input. Whether to use, as a lesion patch image, only a real medical image, both the real medical image and the deformed real medical image, or all of the real medical image, the deformed real medical image, and a virtual lesion image may be set in various ways, depending on specifications, requirements, design, etc. of a medical image processing apparatus.

According to another embodiment of the disclosure, the first input may include both a random variable and a lesion patch image. The processing 1-1 312 may include both processing for generating the virtual lesion image 314 from a random variable and processing for generating the virtual lesion image 314 from a lesion patch image. During the processing 1-1 312, processing corresponding to the type of the first input may be performed.

The virtual lesion image 314 is a virtual image of a lesion generated by performing the processing 1-1 312. The virtual lesion image 314 may include a lesion region and a background region. The lesion region may correspond to a lesion, and the background region may correspond to a region other than the lesion. The background region has a default value. The virtual lesion image 314 may be generated in a predefined size. The virtual lesion image 314 has a width and a length that are respectively less than those of the normal medical image 320 and the first medical image 318.

During the processing 1-2 316, the first medical image 318 may be generated by receiving the virtual lesion image 314 and the normal medical image 320 as input. The normal medical image 320 may be stored in the predetermined database 340 and read by the processor 220. During the processing 1-2 316, a plurality of first medical images 318 may be generated by synthesizing one virtual lesion image 314 with each of a plurality of normal medical images 320. Due to this configuration, the processing 1-2 316 may be performed to generate a plurality of virtual abnormal medical images from the plurality of normal medical images 320.

The normal medical image 320 is an image corresponding to a predefined region being imaged. For example, the normal medical image 320 may be a chest X-ray image. Although the disclosure is mainly described with respect to an example in which the normal medical image 320, the first medical image 318, and abnormal medical images 342 are chest X-ray images, embodiments of the disclosure are not limited thereto. The normal medical image 320, the first medical image 318, and the abnormal medical images 342 may correspond to medical images of various body parts such as a chest, an abdomen, bones, a head, a breast, etc., or medical images of various modalities.

Furthermore, the normal medical image 320 may have a predefined range of sizes, resolutions, etc. The normal medical image 320 may include metadata containing a patient's gender, body weight, height, biometric information, etc., and some or all of the metadata may be used for at least one of the processing 1-2 316 or the second processing 330. Furthermore, during the processing 1-2 316, some or all of the metadata included in the normal medical image 320 may be written to metadata associated with the first medical image 318.

During the second processing 330, it is determined, based on an abnormal medical image 344, whether the first medical image 318 is a real image. The abnormal medical image 344 may be stored in a predetermined database 340 and may be used for the second processing 330. In the second processing 330, the abnormal medical image 344 may be selected randomly or according to a predetermined criterion. One or a plurality of abnormal medical images 344 may be used in the second processing 330.

According to an embodiment of the disclosure, during the second processing 330, the abnormal medical image 344 may be selected based on conditions for synthesizing the virtual lesion image 314. For example, in the second processing 330, the abnormal medical image 344 including a lesion at a similar position to a lesion in the virtual lesion image 314 may be selected from among the abnormal medical images 342, based on a synthesis position from among the conditions for synthesizing the virtual lesion image 314

According to an embodiment of the disclosure, in the second processing 330, the abnormal medical image 344 may be selected based on information related to the lesion in the virtual lesion image 314. For example, the second processing 330 may be performed to select, from among the abnormal medical images 342, the abnormal medical image 344 including a lesion of a similar type and size to a lesion synthesized in the first medical image 318.

According to an embodiment of the disclosure, during the second processing 330, the abnormal medical image 344 may be selected based on information related to a patient in the normal medical image 320. For example, in the second processing 330, the abnormal medical image 344 of a patient of a similar age, bodyweight, height, race, etc., to those of the patient in the normal medical image 320 may be selected from among the abnormal medical images 342.

According to an embodiment of the disclosure, in the second processing 330, the abnormal medical image 344 may be selected based on image data regarding the normal medical image 320. For example, in the second processing 330, the abnormal medical image 344 having a high similarity in an anatomical structure to that in the normal medical image 320 may be selected from among the abnormal medical images 342.

According to an embodiment of the disclosure, in the second processing 330, it is determined, based on the abnormal medical image 344, whether the first medical image 318 is a real medical image. In the second processing 330, an evaluation value corresponding to a result of comparison between the abnormal medical image 344 and the first medical image 318 may be calculated in order to determine whether the first medical image 318 is a real medical image. The evaluation value may be calculated using a predefined algorithm or at least one network. The evaluation value may be calculated by using various determination methods, such as determination using similarity between images, determination using characteristics of image data, determination using image characteristics of an area surrounding a lesion region, etc., or a combination of the various determination methods. For example, in the second processing 330, image characteristics of an area surrounding a boundary of a lesion region in the first medical image 318 are detected and then compared with image characteristics in the abnormal medical image 344 that is a real medical image to determine whether the image characteristics are similar to each other. When the image characteristics of the area surrounding the boundary of the lesion region in the first medical image 318 are similar to those in the abnormal medical image 344, the first medical image 318 is determined to be a real medical image or to have a high probability of being the real medical image. Otherwise, when the image characteristics of the surrounding area in the first medical image 318 are not similar to those in the abnormal medical image 344, the first medical image 318 is not determined to be a real medical image or is determined to have a low probability of being a real medical image.

After the evaluation value is calculated, in the second processing 330, it is determined whether the first medical image 318 is a real medical image by comparing the evaluation value with a specific reference value. In the second processing 330, a determination result value indicating whether the first medical image 318 is a real medical image is generated and output. According to an embodiment of the disclosure, a discrimination algorithm included in a GAN algorithm may be used in the second processing 330.

The processor 220 trains a neural network used in the first processing 310 based on the determination result value output in the second processing 330. In the first processing 310, at least one neural network may be used in either or both of the processing 1-1 312 and the processing 1-2 316. The processor 220 may train the neural network 230 based on the determination result value by performing operations such as defining a layer in at least one neural network used in the first processing 310, defining a node in a layer, defining attributes of a node, defining a weight between nodes, defining a connection relation between nodes, etc. The processor 220 may train the at least one neural network used in the first processing 310 by using, as training data, the determination result value and at least one of the first input, a condition for generating the virtual lesion image 314, which is used in the processing 1-1 312, the virtual lesion image 314, the normal medical image 320, a synthesis condition used in the processing 1-2 316, the first medical image 318, the abnormal medical image 344, or a combination thereof.

Furthermore, the processor 220 trains at least one neural network in the second processing 330 based on the determination result value output in the second processing 330. The processor 220 may train the neural network 230 based on the determination result value by performing operations such as defining a layer in at least one neural network used in the second processing 330, defining a node in a layer, defining attributes of a node, defining a weight between nodes, defining a connection relation between nodes. etc. The processor 220 may train the at least one neural network used in the second processing 330 by using, as training data, the determination result value and at least one or a combination of the first input, a condition for generating the virtual lesion image 314, which is used in the processing 1-1 312, the virtual lesion image 314, the normal medical image 320, a synthesis condition used in the processing 1-2 316, the first medical image 318, a condition for selecting the abnormal medical image 344, or the abnormal medical image 344.

Training of a neural network used in the first or second processing 310 or 330 may be performed using various learning algorithms such as a learning algorithm used in a GAN technique.

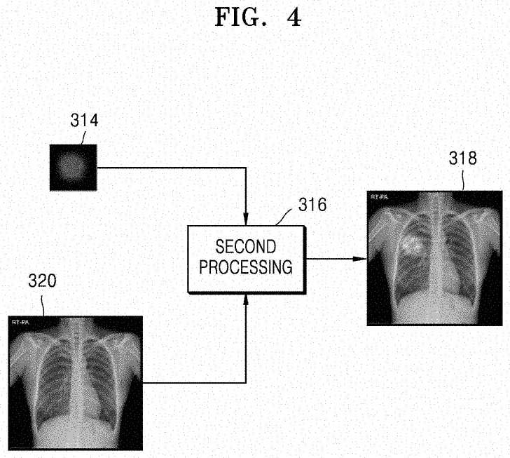

FIG. 4 is a diagram for explaining a procedure for performing processing 1-2 316 according to an embodiment of the disclosure.

In the processing 1-2 316, a first medical image is generated by receiving a virtual lesion image 314 and a normal medical image 320 as input. In the processing 1-2 316, the first medical image 318 is generated by synthesizing the virtual lesion image 314 with the normal medical image 320. In the processing 1-2 316, a condition for synthesizing the virtual lesion image 314 with the normal medical image 320 is determined. The condition for synthesizing the normal medical image 320 with the virtual lesion image 314 may be determined based on information related to a lesion in the virtual lesion image 314, image data regarding the virtual lesion image 314, information related to a patient in the normal medical image 320, image data regarding the normal medical image 320, a preset synthesis condition, a preset rule or logic, etc. The processing 1-2 316 may be performed using a predefined algorithm or at least one neural network according to an embodiment of the disclosure.

The condition for synthesizing the virtual lesion image 314 may include a position in the normal medical image 320 into which the virtual lesion image 314 is to be inserted, a magnification ratio to be applied to the virtual lesion image 314, a condition for processing a region corresponding to a boundary of a lesion region in the virtual lesion image 314, a weight related to synthesis of the virtual lesion image 314 and the normal medical image 320, a synthesis method, etc. The position into which the virtual lesion image 314 is to be inserted may be determined based on at least one of information about an anatomical structure in the normal medical image 320, information related to a lesion in the virtual lesion image 314, image data regarding the virtual lesion image 314, or a combination thereof. The condition for processing the region corresponding to the boundary of the lesion region in the virtual lesion image 314 is a condition as to how to process an edge of a lesion for image synthesis. For example, the condition for processing the region corresponding to the boundary of lesion region includes a condition for smoothing the edge of the lesion. The weight related to synthesis of the virtual lesion image 314 and the normal medical image 320 may include a weighting condition applied as the synthesis proceeds from a center of the lesion region toward its edge. The weighting condition means weights assigned to the virtual lesion image 314 and the normal medical image 320. The synthesis method refers to a method of calculating pixel values used when synthesizing the virtual lesion image 314 with the normal medical image 320, etc. For example, the synthesis method may include image linear summation, convolution, etc.

According to an embodiment of the disclosure, by applying a plurality of synthesis conditions to one virtual lesion image and one normal medical image 320, a plurality of first medical images 318 may be generated from the one virtual lesion image 314 and the one normal medical image 320. For example, in the processing 1-2 316, a plurality of first medical images 318 may be generated by applying a plurality of synthesis positions to the virtual lesion image 314. As another example, in the processing 1-2 316, a plurality of first medical image 318 may be generated by applying a plurality of synthesis methods to the virtual lesion image 314.

According to an embodiment of the disclosure, the virtual lesion image 314 has a lower resolution than that of the normal medical image 320 and the first medical image 318. For example, the virtual lesion image 314 may have a resolution of 70*70, while the normal medical image 320 and the first medical image 318 may have a resolution of 3000*3000. According to embodiments of the disclosure, by sequentially performing processing 1-1 for generating a virtual lesion image only on the virtual lesion image, generating the virtual lesion image, and performing processing 1-2 that is separate processing to synthesize the virtual lesion image with the normal medical image, it is possible to improve the quality of the virtual lesion image and the first medical image and obtain a more natural first medical image.

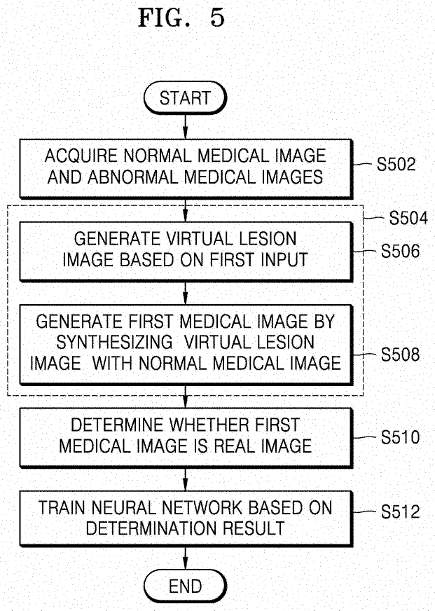

FIG. 5 is a flowchart of a medical image processing method according to an embodiment of the disclosure.

According to embodiments of the disclosure, a medical image processing method may be performed by various types of electronic devices including a processor and a storage. The present specification focuses on an embodiment of the disclosure in which a medical image processing apparatus according to the disclosure performs a medical image processing method according to the disclosure. Thus, embodiments of the disclosure described with respect to a medical image processing apparatus may be applied to a medical image processing method, and embodiments of the disclosure described with respect to a medical image processing method may be applied to embodiments of the disclosure described with respect to a medical image processing apparatus. Although it has been described that medical image processing methods according to embodiments of the disclosure are performed by a medical image processing apparatus according to the disclosure, embodiments of the disclosure are not limited thereto, and the medical image processing methods may be performed by various types of electronic devices.

First, a medical image processing apparatus acquires a normal medical image and an abnormal medical image (S502). The normal and abnormal medical images may be acquired from a predetermined storage, database, or external device.

Next, the medical image processing apparatus performs first processing for generating a first medical image based on a first input (S504). The first input may be a random variable or lesion patch image. The medical image processing apparatus generates a virtual lesion image based on the first input (S506). The virtual lesion image has a preset resolution. Then, the medical image processing apparatus generates a first medical image by synthesizing the virtual lesion image with the normal medical image (S508). As described above, synthesis of the virtual lesion image and the normal medical image includes synthesizing the virtual lesion image with the normal medical image by determining a synthesis condition. Synthesis of the virtual lesion image and the normal medical image may be performed via processing by a preset logic, or may be performed using a trained neural network.

The medical image processing apparatus performs second processing for determining whether the first medical image is a real image based on the abnormal medical image (S510). In the second processing, an evaluation value may be calculated by determining whether the first medical image is a real medical image, and a determination result value may be output. As described above, the second processing may be performed using a predefined algorithm or at least one neural network. Furthermore, in the second processing, one or a plurality of abnormal medical images may be used. In addition, as described above, the abnormal medical image may be selected randomly or according to a predetermined criterion.

The medical image processing apparatus trains, based on a determination result, at least one neural network used in the first processing (S504) and the second processing (S510) (S512). Training of the neural network may be performed using various methods, as described above with reference to FIG. 3.

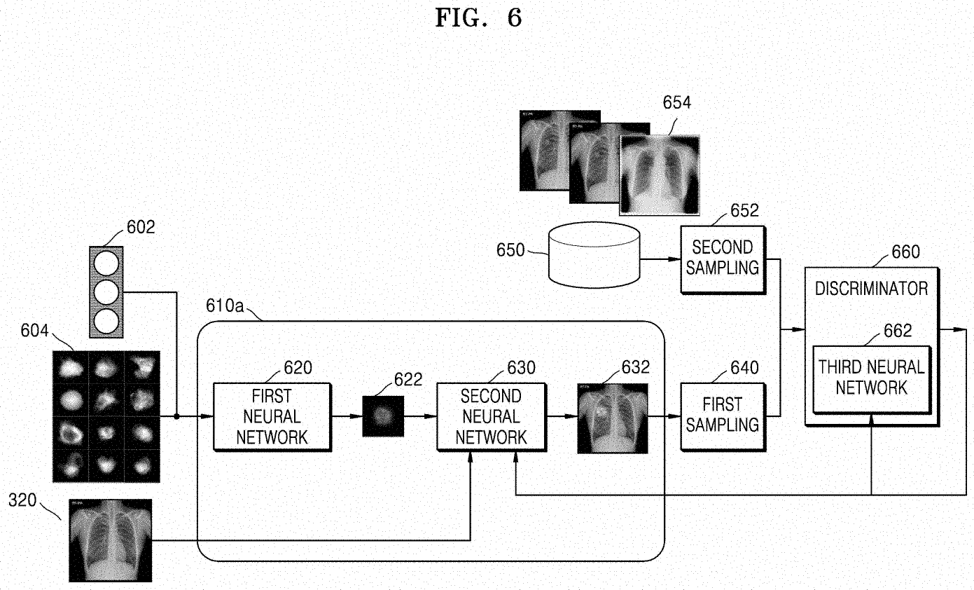

FIG. 6 illustrates structures of the processor 220 and the neural network 230, according to an embodiment of the disclosure.

Referring to FIGS. 2, 3, and 6, according to an embodiment of the disclosure, the processing 1-1 312 and the processing 1-2 316 may be respectively performed using first and second neural networks 620 and 630, and the second processing 330 may be performed by a discriminator 660 including a third neural network 662. The first through third neural networks 620, 630, and 662 may correspond to the neural network 230 provided inside or outside the medical image processing apparatus 200. Each of the first through third neural networks 620, 630, and 662 may be an independent neural network and corresponds to a neural network having defined therein at least one layer, at least one node, and a weight between nodes.

In first processing 610a, a first medical image 632 is generated and output by receiving a first input (602, 604) and a normal medical image 320. The first input may include a random variable 602, a lesion patch image 604, or both the random variable 602 and the lesion patch image 604. The lesion patch image 604 may have a predefined resolution.

The first neural network 620 receives the first input to generate a virtual lesion image 622. The first neural network 620 may define a shape and size of a lesion and pixel values of a lesion region in the virtual lesion image 622. At least one attribute related to the lesion may correspond to a layer or node in the first neural network 620. For example, a lesion shape, a lesion size, pixel values in a lesion region, etc., may respectively correspond to layers or nodes in the first neural network 620. When the lesion patch image 604 is used as the first input, the first neural network 620 may include at least one layer for identifying characteristics of the lesion patch image 604. The first neural network 620 may be a neural network trained using a large number of training data consisting of a pair of the first input and the virtual lesion image 622. According to an embodiment of the disclosure, the first neural network 620 may be trained using, as training data, the first input, the virtual lesion image 622, and a determination result value from the discriminator 660.

The second neural network 630 may receive the virtual lesion image 622 and the normal medical image 320 to generate the first medical image 632. The second neural network 630 determines a condition for synthesizing the virtual lesion image 622 with the normal medical image 320 and synthesizes the virtual lesion image 622 with the normal medical image 320 to generate and output the first medical image 632. At least one of detection of characteristics of the virtual lesion image 622, detection of characteristics of the normal medical image 320, processing of the virtual lesion image 622, processing of the normal medical image 320, determination of a synthesis condition, performing of an image synthesis operation, postprocessing of a synthesized image, or a combination thereof may correspond to at least one layer or node in the second neural network 630.

The second neural network 630 may be trained by using, as training data, at least one of the virtual lesion image 622, the normal medical image 320, the first medical image 632, a determination result value from the discriminator 660, or a combination thereof. The training may be performed by the processor 220. The second neural network 630 may be trained using various learning algorithms such as a learning algorithm used in a GAN technique. The second neural network 630 may be trained such that a rate at which the first medical image 632 is determined as a real medical image by the discriminator 660 reaches a target rate. For example, the second neural network 630 may be trained until a rate at which the first medical image 632 is determined as a real medical image by the discriminator 660 converges to 99.9%. When a rate at which a determination result value is a value of ‘true’ converges to a target rate, the training of the second neural network 630 may be finished.

The first medical image 632 output from the second neural network 630 may be transmitted to the discriminator 660 via first sampling 640. Furthermore, the discriminator 660 may receive at least one abnormal medical image 654 from a database 650 via second sampling 652. As described above, according to an embodiment of the disclosure, the second sampling 652 may be performed to sample the at least one abnormal medical image 654 randomly or according to a predetermined criterion.

The discriminator 660 determines whether the first medical image 632 is a real medical image based on the at least one abnormal medical image 654 by using the third neural network 662. The third neural network 662 may perform processing for extracting characteristics of the first medical image 632, processing for extracting characteristics of a lesion region in the first medical image 632, processing for extracting characteristics of the at least one abnormal medical image 654, or processing for determining whether the first medical image 632 is a real medical image, and each processing may correspond to at least one layer or at least one node in the third neural network 662. Furthermore, the third neural network 662 may output a determination result value indicating a result of determining whether the first medical image 632 is a real medical image. For example, the determination result value may correspond to the probability that the first medical image 632 is a real medical image or a value representing ‘true’ or ‘false’.

According to an embodiment of the disclosure, the discriminator 660 may determine whether the first medical image 632 is a real medical image by using at least some of metadata associated with the first medical image 632 or metadata associated with the abnormal medical image 654. The third neural network 662 may receive the at least some of metadata associated with the first medical image 632 or metadata associated with the abnormal medical image 654. For example, the discriminator 660 may use at least one or a combination of a patient's age, gender, height, body weight, or race contained in metadata for determination.

The third neural network 662 is trained using at least one or a combination of the first medical image 632, the abnormal medical image 654, or a determination result value from the discriminator 660. Furthermore, according to an embodiment of the disclosure, at least one or a combination of the first input, the normal medical image 320, the metadata associated with the normal medical image 320, or the metadata associated with the abnormal medical image 654 may be used as training data for the third neural network 662.

According to an embodiment of the disclosure, an architecture and a training operation of the third neural network 662 may be implemented using an architecture and a training operation of a discriminator in a GAN technique.

According to an embodiment of the disclosure, the processor 220 may perform training on the second and third neural networks 630 and 662 based on a determination result value while not performing on the first neural network 620. Thus, the second and third neural networks 630 and 662 may each be modified or updated due to the training based on the determination result value. The first neural network 620 may correspond to a pre-trained neural network and may be excluded from being a candidate for training based on the determination result value.

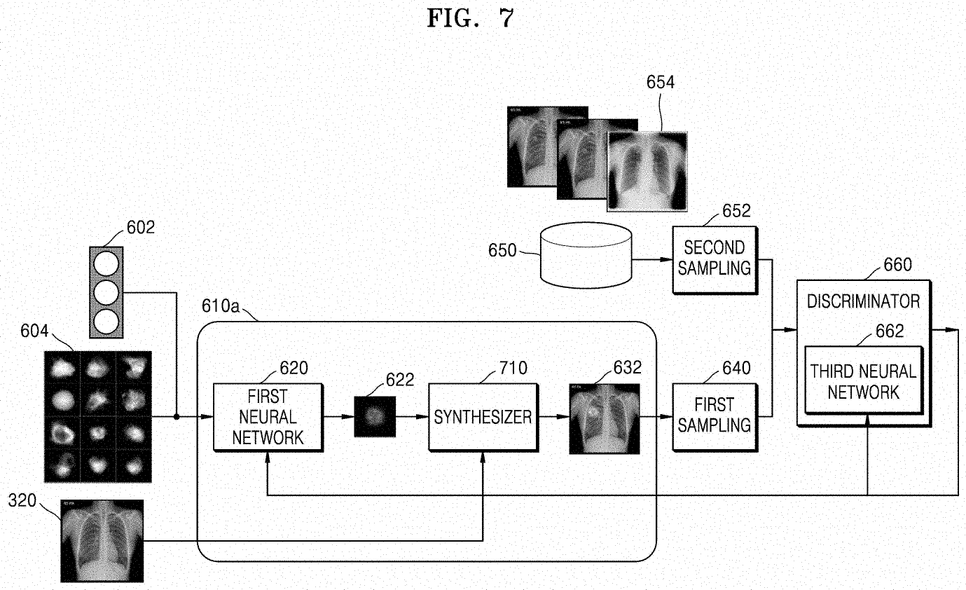

FIG. 7 illustrates structures of the processor 220 and the neural network 230, according to an embodiment of the disclosure.

According to an embodiment of the disclosure, the processing 1-1 312 may be performed using a first neural network 620, the processing 1-2 316 may be performed by a synthesizer 710 for performing a predefined logic, and the second processing 330 may be performed by a discriminator 660 including a third neural network 662. The first and third neural networks 620 and 662 may correspond to the neural network 230 provided inside or outside the medical image processing apparatus 200. Each of the first and third neural networks 620 and 662 may be an independent neural network and corresponds to a neural network having defined therein at least one layer, at least one node, and a weight between nodes.

Descriptions that are already provided above with respect to FIG. 6 are omitted herein, and only a difference is described.

According to an embodiment of the disclosure, the synthesizer 710 may synthesize a virtual lesion image 622 with a normal medical image 320 according to a predefined logic to generate a first medical image 632. The synthesizer 710 may determine a condition for synthesizing the virtual lesion image 622 with the normal medical image 320, based on a predetermined criterion. According to an embodiment of the disclosure, the synthesizer 710 may determine a synthesis condition based on a user input received via an inputter (not shown). According to an embodiment of the disclosure, the synthesizer 710 may generate a plurality of first medical images 632 based on the virtual lesion image 622 and the normal medical image 320 by using a prestored combination of various synthesis conditions or generating a combination thereof. To achieve this, algorithms such as a look-up table that defines a combination of various synthesis conditions may be used.

The synthesizer 710 may perform at least one or a combination of detection of characteristics of the virtual lesion image 622, detection of characteristics of the normal medical image 320, processing of the virtual lesion image 622, processing of the normal medical image 320, determination of a synthesis condition, performing of an image synthesis operation, or postprocessing of a synthesized image. The synthesizer 710 may perform each operation by executing at least one instruction defined to perform the operation.

The synthesizer 710 may synthesize a lesion in a predefined region during synthesis of the lesion in the normal medical image 320 and output information about a position where the lesion has been synthesized together with the first medical image 632. For example, the synthesizer 710 may arrange a lesion in a lung cancer in a lung region and output a position of the lesion as metadata associated with the first medical image 632.

The processor 220 may train the first and third neural networks 620 and 662 based on a determination result value from the discriminator 660. According to an embodiment of the disclosure, the synthesizer 710 may not include a neural network and be excluded from being a candidate to be trained. The first and third neural networks 620 and 662 may each be modified or updated due to the training based on the determination result value. According to an embodiment of the disclosure, because the first neural network 620 learns only a type of a lesion, a difficulty level for training the first neural network may be lowered.

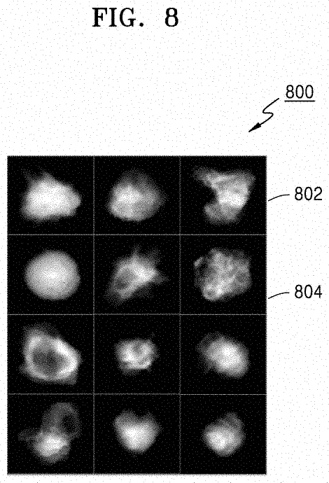

FIG. 8 illustrates a form of a first input according to an embodiment of the disclosure.

The first input may correspond to a plurality of lesion patch images 802 and 804 shown in FIG. 8. The lesion patch images 802 and 804 may be images in which a lesion type, a lesion size, a lesion shape, or a pixel value in a lesion region is defined. The lesion patch images 802 and 804 may be extracted from a real medical image. According to an embodiment of the disclosure, the lesion patch images 802 and 804 may be generated using predetermined processing for generating a lesion image.

According to an embodiment of the disclosure, the lesion patch images 802 and 804 in a set 800 of lesion patch images may be sequentially input as a first input for first processing, or the set 800 of lesion patch images may be input as the first input for the first processing.

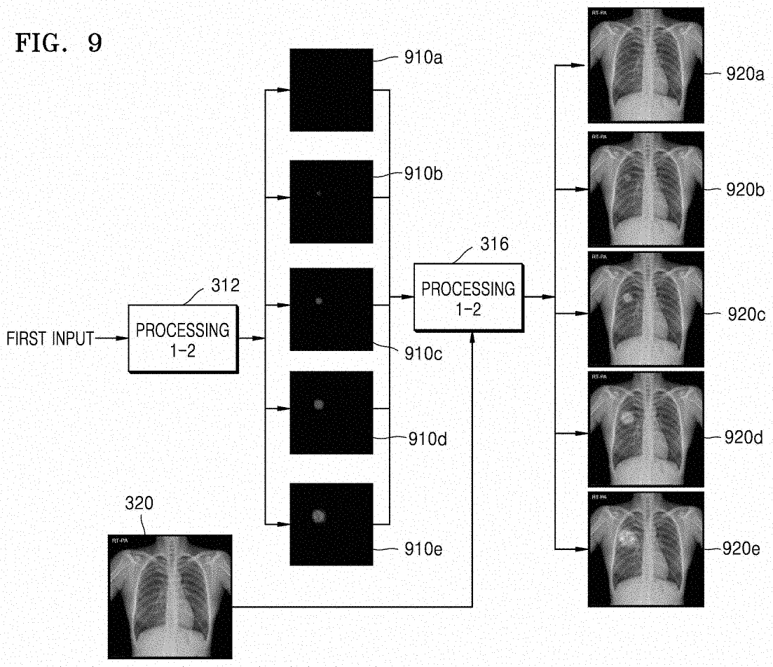

FIG. 9 illustrates a process of generating a first medical image, according to an embodiment of the disclosure.

According to an embodiment of the disclosure, a plurality of first medical images 920a through 920e based on a first input and a normal medical image 320.

In processing 1-1 312, a plurality of virtual lesion images 910a through 910e are generated based on the first input. The number of the virtual lesion images 910a through 910e may be determined in various ways according to an embodiment of the disclosure. In the virtual lesion images 910a through 910e, a lesion shape, a lesion size, or pixel values in a lesion region may be determined in various ways according to an embodiment of the disclosure. In the processing 1-1 312, the number of virtual lesion images 910a through 910e, a shape and a size of a lesion therein, etc. may be determined based on preset conditions.

According to an embodiment of the disclosure, a first neural network used in the processing 1-1 312 may determine the number of virtual lesion images 910a through 910e generated based on the first input and a shape and a size of a lesion therein. The first neural network may include at least one layer or node corresponding to processing for determining the number of virtual lesion images, a shape of a lesion therein, or a size of the lesion. For example, when a virtual lesion image corresponding to a tumor is generated based on the first input, the first neural network may generate a plurality of virtual lesion images showing the degree of progression of a cancer. As another example, when a virtual lesion image corresponding to a pneumothorax is generated based on the first input, the first neural network may generate a plurality of virtual lesion images to which different types and sizes of chest wall injury are applied.

In processing 1-2 316, the plurality of first medical images 920a through 920e may be generated by respectively synthesizing the virtual lesion images 910a through 910e generated via the processing 1-1 312 with the normal medical image 320. In the processing 1-2 316, different synthesis conditions may be respectively applied to the virtual lesion images 910a through 910e. Furthermore, in the processing 1-2 316, a synthesis condition for another virtual lesion image may be determined by referring to a synthesis condition determined for one of the virtual lesion images 910a through 910e. For example, in the processing 1-2 316, a synthesis condition for the virtual lesion image 910b may be determined based on a synthesis position, a synthesis method, etc. determined for the virtual lesion image 910a.

According to an embodiment of the disclosure, in the processing 1-2 316, the plurality of first medical images 920a through 920e may be generated by receiving the virtual lesion images 910a through 910e and the normal medical images 320. For example, in the processing 1-2 316, N*M first medical images may be generated by receiving N virtual lesion images and M normal medical images wherein N and M are natural numbers.

According to an embodiment of the disclosure, the plurality of first medical images 920a through 920e may correspond to medical images showing the progression of disease. For example, the plurality of first medical images 920a through 920e may correspond to medical images showing progression of lung cancer such as four stages of the lung cancer, i.e., stages 1 to 4.

According to an embodiment of the disclosure, the plurality of first medical images 920a through 920e may respectively correspond to medical images in which a size, a position, etc., of a disease region are set differently. For example, the plurality of first medical images 920a through 920e may correspond to medical images in which lung cancer cells are arranged in the left lung, the right lung, etc.

According to the embodiment of disclosure described with reference to FIG. 9, it is possible to significantly increase the efficiency and speed of generation of training data by simultaneously generating various first medical images.

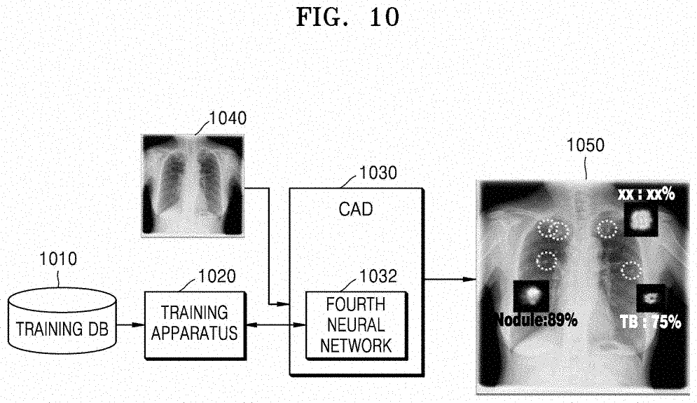

FIG. 10 illustrates a training apparatus 1020 and an auxiliary diagnostic device according to an embodiment of the disclosure.

According to an embodiment of the disclosure, when a rate at which a first medical image is determined as a real medical image based on a determination result value reaches a target rate and training of the neural network 230 described with reference to FIGS. 2, 3, 6, and 7 is finished, a large number of training data corresponding to a medical image including a lesion may be generated by the medical image processing apparatus 200. The medical image processing apparatus 200 may generate a large number of training data by using a large number of first inputs and a large number of normal medical images. The training data generated by the medical image processing apparatus 200, i.e., a large number of first medical images, are stored in a training database (DB) 1010. The training data may include image data regarding a first medical image, a position, type, or shape of a lesion, etc. The training apparatus 1020 may train a fourth neural network 1032 used by an auxiliary diagnostic device 1030 for identifying information about a lesion or disease in a medical image by using training data stored in the training DB 1010.

The auxiliary diagnostic device 1030 may receive a real medical image 1040 and detect a disease or lesion in the real medical image 1040 to generate an auxiliary diagnostic image 1050 showing information about the disease or lesion. The auxiliary diagnostic device 1030 may correspond to a computer-aided detection or diagnosis (CAD) system. The auxiliary diagnostic device 1030 may use the fourth neural network 1032 to generate information such as a position, size, and shape of a disease region or lesion, severity of disease, a probability of being a lesion, etc. and display the information. The fourth neural network 1032 may be included in the auxiliary diagnostic device 1030 or be provided in an external device such as a server.

The training apparatus 1020 may train the fourth neural network 1032 by using training data stored in the training DB 1010. The training apparatus 1020 may train the fourth neural network 1032 by acquiring, preprocessing, and selecting training data, and update or modify the fourth neural network 1032 by evaluating the trained fourth neural network 1032. The training apparatus 1020 may train the fourth neural network 1032 by determining a layer in the fourth neural network 1032, a node structure, number of nodes, attributes of a node, a weight between nodes, a relation between nodes, etc., and train the fourth neural network 1032.

The fourth neural network 1032 may perform processing such as extraction of at least one characteristic of a medical image, detection of a disease or lesion, determination of a disease or lesion region, extraction of a probability of being a disease or lesion, etc. Each processing may correspond to at least one layer or at least one node.

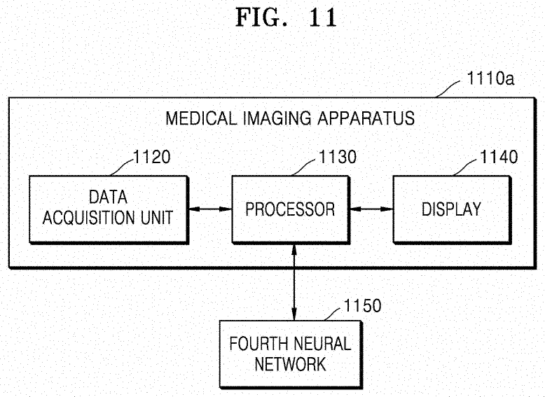

FIG. 11 is a block diagram of a configuration of a medical imaging apparatus 1110a according to an embodiment of the disclosure.

According to an embodiment of the disclosure, the auxiliary diagnostic device 1030 described above may be included in the medical imaging apparatus 1110a. The medical imaging apparatus 1110a may include hardware, software, or a combination thereof used to implement auxiliary diagnosis by the auxiliary diagnostic device 1030. The medical imaging apparatus 1110a may use a fourth neural network 1150 trained in the manner described with reference to FIG. 10.

The medical imaging apparatus 1110a may correspond to any one of medical apparatuses of various imaging modalities, such as an X-ray imaging apparatus, a CT system, an MRI system, or an ultrasound system. The medical imaging apparatus 1110a may include a data acquisition unit 1120, a processor 1130, and a display 1140.

The data acquisition unit 1120 acquires raw data for a medical image. According to an embodiment of the disclosure, the data acquisition unit 1120 corresponds to a communicator for receiving raw data from an external device. According to another embodiment of the disclosure, the data acquisition unit 1120 may correspond to the X-ray radiation device 110 and the X-ray detector 195 of the X-ray apparatus 100. According to another embodiment of the disclosure, the data acquisition unit 1120 may correspond to a scanner in a CT or MRI system for scanning an object to acquire raw data. According to another embodiment of the disclosure, the data acquisition unit 1120 may correspond to an ultrasound probe of an ultrasound system.

The processor 1130 generates a medical image from raw data acquired by the data acquisition unit 1120. According to an embodiment of the disclosure, the processor 1130 detects information about a disease or lesion in a medical image by performing auxiliary diagnosis on the generated medical image. The processor 1130 may use the trained fourth neural network 1150 to perform auxiliary diagnosis. The fourth neural network 1150 may receive a medical image from the processor 1130 to identify information about a disease or lesion and output the information to the processor 1130. The processor 1130 generates the auxiliary diagnostic image (1050 of FIG. 10) showing the information about a disease or lesion and displays the auxiliary diagnostic image 1050 on the display 1140.

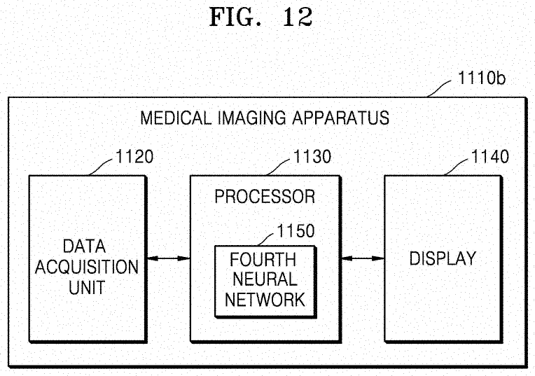

FIG. 12 is a block diagram of a configuration of a medical imaging apparatus 1110b according to an embodiment of the disclosure.

According to an embodiment of the disclosure, the medical imaging apparatus 1110b may include a trained fourth neural network 1150. A processor 1130 of the medical imaging apparatus 1110b generates the auxiliary diagnostic image 1050 from a medical image by using the fourth neural network 1150 and displays the auxiliary diagnostic image 1050 on a display 1140.

The embodiments of the disclosure may be implemented as a software program including instructions stored in computer-readable storage media.

A computer may refer to a device capable of retrieving instructions stored in the computer-readable storage media and performing operations according to embodiments of the disclosure in response to the retrieved instructions, and may include tomographic image processing apparatuses according to the embodiments of the disclosure.

The computer-readable storage media may be provided in the form of non-transitory storage media. In this case, the term ‘non-transitory’ only means that the storage media do not include signals and are tangible, and the term does not distinguish between data that is semi-permanently stored and data that is temporarily stored in the storage media.

In addition, medical image processing apparatuses or methods according to embodiments of the disclosure may be included in a computer program product when provided. The computer program product may be traded, as a commodity, between a seller and a buyer.

The computer program product may include a software program and a computer-readable storage medium having stored thereon the software program. For example, the computer program product may include a product (e.g. a downloadable application) in the form of a software program electronically distributed by a manufacturer of a tomographic image processing apparatus or through an electronic market (e.g., Google Play Store™, and App Store™). For such electronic distribution, at least a part of the software program may be stored on the storage medium or may be temporarily generated. In this case, the storage medium may be a storage medium of a server of the manufacturer, a server of the electronic market, or a relay server for temporarily storing the software program.

In a system consisting of a server and a terminal (e.g., an X-ray imaging system), the computer program product may include a storage medium of the server or a storage medium of the terminal. Alternatively, in a case where a third device (e.g., a smartphone) is connected to the server or terminal through a communication network, the computer program product may include a storage medium of the third device. Alternatively, the computer program product may include a software program itself that is transmitted from the server to the terminal or the third device or that is transmitted from the third device to the terminal.

In this case, one of the server, the terminal, and the third device may execute the computer program product to perform methods according to embodiments of the disclosure. Alternatively, two or more of the server, the terminal, and the third device may execute the computer program product to perform the methods according to the embodiments of the disclosure in a distributed manner.

For example, the server (e.g., a cloud server, an AI server, or the like) may run the computer program product stored therein to control the terminal communicating with the server to perform the methods according to the embodiments of the disclosure.

As another example, the third device may execute the computer program product to control the terminal communicating with the third device to perform the methods according to the embodiments of the disclosure. As a specific example, the third device may remotely control the X-ray imaging system to emit X-rays toward an object and generate an image of an inner area of the object based on information about radiation that passes through the object and is detected by the X-ray detector.

As another example, the third device may execute the computer program product to directly perform the methods according to the embodiments of the disclosure based on a value received from an auxiliary device. As a specific example, the auxiliary device may emit X-rays toward an object and acquire information about the radiation that passes through the object and is detected. The third device may receive information about the radiation detected by the auxiliary device and generate an image of an inner area of the object based on the received information about the radiation.

In a case where the third device executes the computer program product, the third device may download the computer program product from the server and execute the downloaded computer program product. Alternatively, the third device may execute the computer program product that is pre-loaded therein to perform the methods according to the embodiments of the disclosure.

According to embodiments of the disclosure, an apparatus and method of generating high quality medical images to be used as training data may be provided.

Furthermore, according to embodiments of the disclosure, it is possible to generate various medical images corresponding to disease progression stages, which are to be used as training data.

Furthermore, a training apparatus for performing training with generated training data and a medical imaging apparatus employing a model trained using the generated training data may be provided.

While one or more embodiments of the disclosure have been described with reference to the figures, it will be understood by those of ordinary skill in the art that various changes in form and details may be made therein without departing from the spirit and essential characteristics of the disclosure as defined by the following claims. Accordingly, the above embodiments of the disclosure are examples only and are not limiting.

What is claimed is:

1. A medical image processing apparatus comprising:
a data acquisition unit configured to acquire at least one normal medical image and at least one abnormal medical image; and
one or more processors configured to:
perform, using at least one neural network, first processing that includes generating at least one virtual lesion image based on at least one first input, and generating at least one first medical image by synthesizing the at least one virtual lesion image with the at least one normal medical image,
perform second processing that includes determining whether the at least one first medical image is a real image, based on the at least one abnormal medical image, and
train a neural network of the at least one neural network used in the first processing, based on a result of the second processing.

2. The medical image processing apparatus of claim 1, wherein the at least one first input comprises a random variable input.

3. The medical image processing apparatus of claim 1, wherein the at least one first input comprises a lesion patch image.

4. The medical image processing apparatus of claim 1, wherein
the at least one neural network includes
a first neural network used by the first processing in the generating the at least one virtual lesion image based on at least one first input, and
a second neural network used by the first processing in the generating the at least one first medical image by synthesizing the at least one virtual lesion image with the at least one normal medical image, and
the one or more processors are further configured to train the second neural network based on the result of the second processing.

5. The medical image processing apparatus of claim 1, wherein
the at least one neural network includes
a first neural network used by the first processing in the generating the at least one virtual lesion image based on at least one first input, and
a second neural network used by the first processing in the generating the at least one first medical image by synthesizing the at least one virtual lesion image with the at least one normal medical image, and
the one or more processors are further configured to train the first neural network based on the result of the second processing.

6. The medical image processing apparatus of claim 1, wherein the at least one normal medical image and the at least one abnormal medical image are respectively chest X-ray images.

7. The medical image processing apparatus of claim 1, wherein
the at least one neural network includes
a first neural network used by the first processing in the generating the at least one virtual lesion image based on at least one first input, and
a second neural network used by the first processing in the generating the at least one first medical image by synthesizing the at least one virtual lesion image with the at least one normal medical image, and
the one or more processors are further configured to use a third neural network to perform the second processing, and to train the third neural network based on the result of the second processing.

8. The medical image processing apparatus of claim 1, wherein the first processing includes generating a plurality of virtual lesion images corresponding to different disease progression states, based on the at least one first input, and generating a plurality of first medical images corresponding to the different disease progression states by respectively synthesizing the plurality of virtual lesion images with the at least one normal medical image.

9. The medical image processing apparatus of claim 1, wherein the first processing includes generating a plurality of first medical images by respectively synthesizing one of the at least one virtual lesion image with a plurality of different normal medical images.

10. The medical image processing apparatus of claim 1, wherein the second processing includes determining whether the at least one first medical image is a real image based on characteristics related to lesion regions respectively in the at least one abnormal medical image and in the at least one first medical image.

11. The medical image processing apparatus of claim 1, wherein the one or more processors are further configured to select the at least one abnormal medical image to be used in the second processing, based on information about the at least one first medical image generated in the first processing.

12. The medical image processing apparatus of claim 1, wherein a resolution of the at least one virtual lesion image is lower than a resolution of the at least one abnormal medical image and a resolution of the at least one first medical image.

13. The medical image processing apparatus of claim 1, wherein each of the at least one normal medical image and the at least one abnormal medical image is at least one of an X-ray image a computed tomography (CT) image, a magnetic resonance imaging (MRI) image, or an ultrasound image.

14. A training apparatus for training a neural network that generates an auxiliary diagnostic image showing at least one of a lesion position, a lesion type, or a probability of being a lesion by using the at least one first medical image generated by the medical image processing apparatus of claim 1.

15. A medical imaging apparatus for displaying the auxiliary diagnostic image generated using the neural network trained by the training apparatus of claim 14.

16. A medical image processing method comprising:
acquiring at least one normal medical image and at least one abnormal medical image;
performing, using at least one neural network, first processing that includes generating at least one virtual lesion image based on at least one first input, and generating at least one first medical image by synthesizing the at least one virtual lesion image with the at least one normal medical image;
performing second processing that includes determining whether the at least one first medical image is a real image, based on the at least one abnormal medical image; and
training a neural network of the at least one neural network used in the first processing, based on a result of the second processing.

17. The medical image processing method of claim 16, wherein the at least one first input comprises a random variable input.

18. The medical image processing method of claim 16, wherein the at least one first input comprises a lesion patch image.

19. The medical image processing method of claim 16, wherein
the at least one neural network includes
a first neural network used by the first processing in the generating the at least one virtual lesion image based on at least one first input, and
a second neural network used by the first processing in the generating the at least one first medical image by synthesizing the at least one virtual lesion image with the at least one normal medical image, and
the method further comprises training the second neural network based on the result of the second processing.

20. A computer program stored on a recording medium, wherein the computer program comprises at least one instruction that, when executed by a processor, causes a medical image processing method to be performed, the medical image processing method comprising:
acquiring at least one normal medical image and at least one abnormal medical image;
performing, using at least one neural network, first processing that includes generating at least one virtual lesion image based on at least one first input, and generating at least one first medical image by synthesizing the at least one virtual lesion image with the at least one normal medical;
performing second processing that includes determining whether the at least one first medical image is a real image, based on the at least one abnormal medical image; and
training a neural network of the at least one neural network used in the first processing, based on a result of the second processing.
